(19) United States
(12) Patent Application Publication
(10) Pub. No.: US 2012/0087844 A1
(43) Pub. Date: Apr. 12, 2012

(54) ARRANGEMENT TO OPTIMISE TEMPERATURE DISTRIBUTION WITHIN A STERILISATION CHAMBER, AND AUTOCLAVE OBTAINED BY SUCH ARRANGEMENT

(75) Inventor: Daniele Ongaro, Villa Di Serio (Bergamo) (IT)

(73) Assignee: W & H STERILIZATION S.R.L., Brusaporto (Bergamo) (IT)

(21) Appl. No.: 13/265,901

(22) PCT Filed: Apr. 23, 2010
(86) PCT No.: PCT/IB10/51799

§ 371 Date: Dec. 14, 2011

(30) Foreign Application Priority Data
Apr. 24, 2009 (IT) MI2009A000703

(51) Int. Cl. A61L 2/18; B01J 19/00

(52) U.S. Cl. 422/292; 422/310

(57) ABSTRACT

An autoclave for the sterilization of small medical objects includes at least one sterilization chamber with a heated wall and into which process water to be vaporized may be injected, a deflector plate being housed in the chamber, apt to define a gap with respect to the inner chamber wall.

### FIELD OF THE INVENTION

The present invention concerns an arrangement suited to optimise and even out temperature distribution within an autoclave for the sterilisation of medical instruments, in particular for dental use.

### BACKGROUND ART In medical surgeries, in particular in dental surgeries, the use of small-sized autoclaves for sterilising small instruments or medical tools has been known for a long time.

An example autoclave of this type is described for example in EP 992,247.

The most popular surgery autoclaves usually have a cylindrical-shaped sterilisation chamber having a volume in the order of 15-25 litres, whereto high-temperature steam is supplied, directly created in the sterilisation chamber or blown in from an external source or generator.

The known techniques for the generation and delivery of steam for small-sized sterilisers rely mainly to:

1. steam generation directly in the chamber, through a heating element immersed in water within the sterilisation chamber;

2. steam generation within the chamber, through a heating element, arranged outside in contact with the metal wall of the sterilisation chamber, which transfers heat and water injected into the sterilisation chamber;

3. steam generation by means of an external generator and subsequent steam inflow into the sterilisation chamber.

The first described technique, which allows a simple and effective, direct transfer of the electrical resistance heat to the water, highlights technical limitations due to the fact that:

it is necessary to introduce a large amount of water into the sterilisation chamber in order to guarantee resistance immersion during operation;

it requires the presence of a sensor to detect the presence of water inside the sterilisation chamber, in the absence of which the heating element would be subject to burn;

an inevitable corrosion of the heating element immersed in water exists;

it is difficult to perform ordinary maintenance, for example full cleaning, due to hard-to-reach crevices;

the heating element is rather fragile;

The third described technique advantageously allows to let into the chamber pressurised steam, creating a certain turbulence which ensures temperature evenness within the chamber. However, it has limits linked to the manufacturing, running, design and building costs, since it requires a pressurised container which must meet performance and safety requirements required by laws and possible applicable, currently enforced directives.

The second described technique—which will be referred to in the following of the description—allows to optimise the required amount of water to carry out a sterilisation cycle and does not require an external pressurised container. However, it has technical limitations linked to the sizing difficulty of the heating element which is externally in contact with the sterilisation chamber, with the inevitable consequence of forming—within the sterilisation compartment—localised “hot” volumes of overheated steam and less warm areas; this heating unevenness is to be considered a critical problem, since the sterilisation operation requires to have a reasonable certainty that minimum exposition times to the sterilising agent (steam) are observed for the entire load inserted in the sterilisation chamber.

The Applicant has hence set himself the aim of addressing the negative aspects just cited, trying to exploit instead the positive aspects of a steam generator lying outside the sterilisation chamber, to obtain a sufficiently economic and safe autoclave, despite guaranteeing an even distribution of temperature within the sterilisation chamber.

### SUMMARY OF THE INVENTION

Such result has been achieved by cleverly exploiting thermodynamics concepts which allow the simulation of the presence of a steam generator as a separate component, even though within the sterilisation chamber, through an original arrangement described, in its essential features, in the attached claims.

In particular, according to a first aspect of the invention, an arrangement of a sterilisation chamber for an autoclave is provided, comprising a sterilisation container with a conductive wall and a heating band element, provided with an outer heating element arranged at least in the proximity of a lower area of the container wall, water injection means being further arranged inside said container, wherein a deflector plate is furthermore provided, arranged within the container and at short distance with its internal surface at least in the lower wall portion.

According to a preferred aspect of the invention, the deflector plate has a curvature complementary to that of the container wall.

According to a third aspect, the deflector plate is arranged at a distance from the inner surface of the container between 1 and 3 mm, preferably of about 2 mm.

According to a fourth aspect, the plate is substantially continuous for the entire depth of the container and extends transversally for an arc-of-a-circle proportional to the size of the heating element.

According to a fifth aspect, the deflector plate is complementary and joined to a tray-carrying basket, which may be possibly removed or reintroduced into the autoclave container. In such case, the plate preferably has raised side edges whereto said tray-carrying basked may be fastened. According to a further preferred aspect, the deflector plate is defined by the same thin wall of a tray-carrying basket to be installed inside the sterilisation container.

According to a further aspect of the invention, a tray-carrying basket is provided for an arrangement as detailed above, comprising a side wall substantially shaped like a plate developed on a cylinder and provided with spacer means apt to define a gap between the same side wall of the basket and the inner surface of a sterilisation chamber.

Finally, according to a last aspect, an autoclave for the sterilisation of medical tools is provided, comprising an arrangement as detailed above.

### BRIEF DESCRIPTION OF THE DRAWINGS

Further features and advantages of the arrangement and of the autoclave according to the invention are in any case more evident from the following detailed description of a preferred embodiment of the same, given by way of example and shown in the attached drawings, wherein:

FIG. 1 is a perspective schematic view, which shows a sterilisation chamber in standard conditions;

FIG. 2 is a view similar to FIG. 1 wherein the sterilisation chamber is partly taken up by a deflector according to the invention;

FIG. 3 is a schematic view in a crosswise section, which shows the operation of the arrangement of the invention;

FIG. 4 is a view similar to FIG. 2 wherein the sterilisation chamber is taken up by the deflector integrated with a tray-carrying basket according to a preferred embodiment; and

FIG. 5 is a perspective view of an alternative embodiment of a deflector according to the invention defined by a circular tray-carrying basket.

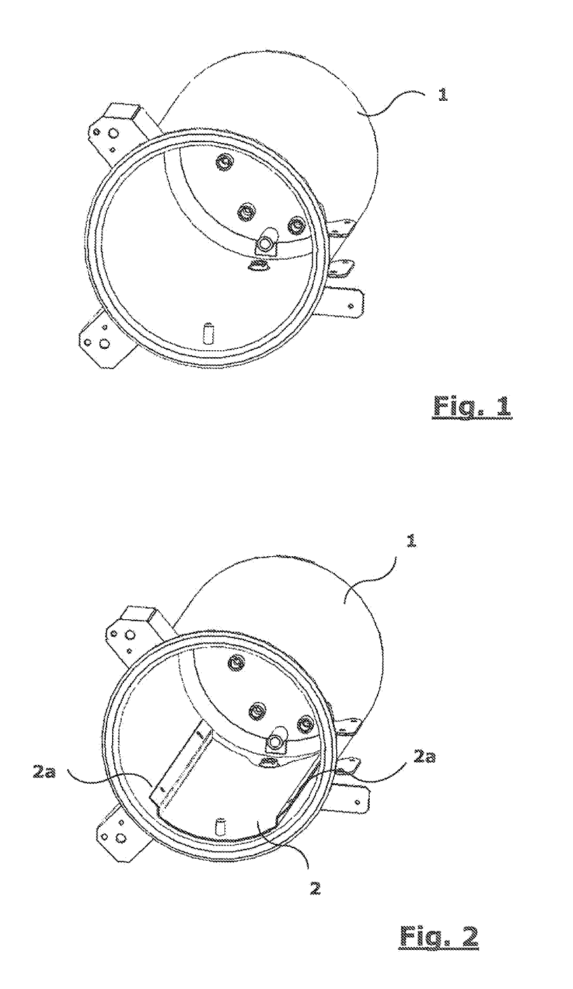

FIG. 1 shows a sterilisation chamber exemplifying a surgery autoclave. Such chamber is substantially shaped as a cylindrical container 1, pressure proof sealable, with metal walls of a suitable thickness, open on the front. As a matter of fact, the cylindrical container is mounted within an autoclave, with the central symmetry axis substantially horizontal, so that its loading/emptying occurs with a horizontal movement through the opening on the front (which represents one of the two bases of the cylindrical shape).

In particular, from the front opening the load may be introduced/removed (typically tools and handpieces for dental use) to be sterilised. The front opening may then be closed, in a sealable and pressure-proof manner, by a door (not shown), generally hinged on one side.

In the lower part and in the back wall (the other base of the cylindrical shape) of the sterilisation chamber, loading and unloading ports of process fluids are provided.

On the outer surface of chamber 1 a heating band (not shown) is furthermore installed, which typically adduces heat to the metal wall by Joule effect, so as to transfer it by conduction to within chamber 1, into which process water is injected.

Typically, the heating band has a heating portion located in the lower portion (because the water introduced into the chamber naturally tends to settle near such wall portion), which is capable of reaching, in a thermostatically controlled way, about 250-300°.

According to the invention, inside the sterilisation chamber, in such lower portion of the wall, a deflector plate 2 is installed arranged in a position at short distance (between 1 and 3 mm, preferably at about 2 mm) with the inner surface of the chamber.

Such plate or deflector, in order to follow at a short distance the inner surface, is curved according to the cylindrical curvature of the wall of chamber 1. In substance, as clearly visible in FIG. 2, deflector 2, so as to be kept at short distance, consists of a thin body of a shape substantially complementary to that of the sterilisation chamber, also compatibly with the introduction of further equipment of the autoclave, such as a support member or a tray basket.

Deflector plate 2 extends across the inner surface of chamber 1 along its entire depth and, transversally, for an arch-of-a-circle proportional to the size of the external heating member.

Deflector plate 2, in the embodiment shown in FIG. 2, is continuous and, preferably, ends laterally in the proximity of the inner wall of the sterilisation chamber, so as to define a slit which makes up the opening towards the chamber of the gap found below plate 2. Preferably, deflector 2 ends laterally with slightly folded, raised step-wise edges 2a, designed to fasten thereto other functional members of the autoclave. In particular, as illustrated in FIG. 4, to raised edges 2a it is possible to constrain—in an integral or detachable manner—a small frame or basket 4 designed to house trays for the load to be sterilized.

In the depth direction, plate 2 is preferably interrupted or notched in correspondence of water loading/unloading ports arranged on the lower wall of chamber 1 (as visible in FIG. 2).

Regardless from the fact that deflector 2 is integral or not with the tray support, the deflector body is preferably removable from the sterilisation container, so that the cleaning operations of the sterilisation chamber may occur in a practical and effective manner, with subject to the temporary removal of the same.

The deflector plate may be made of any material withstanding temperatures of the order of 150° C. (for example aluminium, polymeric materials, stainless steel, and other) and of a suitable mechanical resistance.

The use of a material with a high thermal capacity is preferred, because it contributes to transmit heat to the tray support, contributing through an even temperature distribution to the improvement of the load drying phase performed during the water steam sterilisation processes.

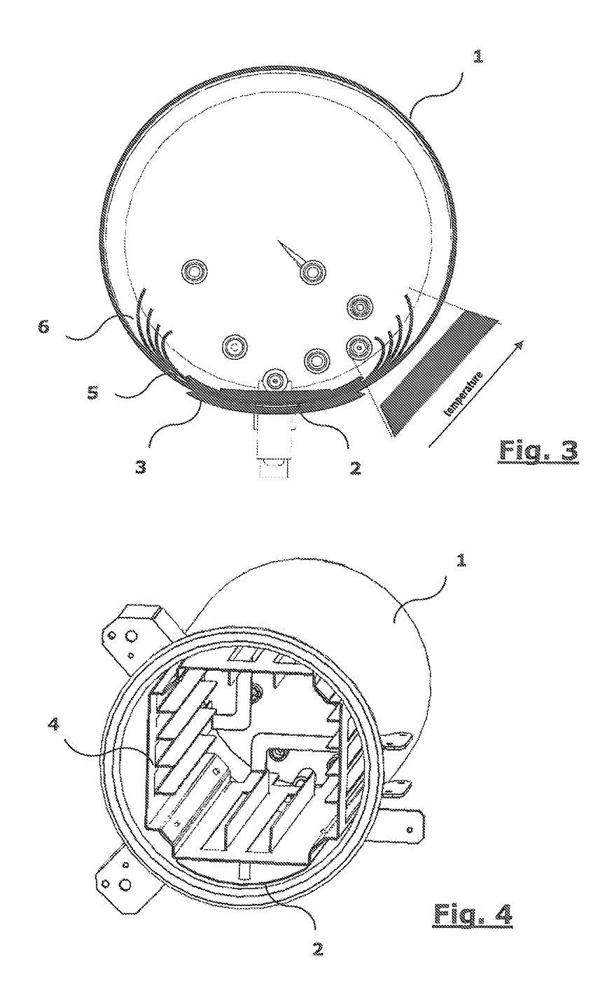

Since deflector plate 2 is arranged at short distance from the wall of chamber 1, as mentioned, in correspondence of the lateral edge thereof it defines a sort of rectilinear slit 5, arranged in the direction of the chamber depth, adjacent to the chamber wall. The steam produced by the water injected in contact with the lower portion of the heated wall of chamber 1 hence tends to remain trapped below the deflector plate, where it is overheated, and hence to come out under pressure from slit 5. Coming out under pressure from slit 5, the steam produces a significant turbulence (shown in FIG. 3 by flows 6), which cooperates effectively to make the temperature inside chamber 1 even.

For such purpose, it is preferable that the raised edge 2a, where provided, detaches sharply from the terminal edge of plate 2, typically with a step, because a smoother connection would tend to reduce the obtainable turbulence level.

It was possible to ascertain that, with the arrangement described above a highly regular and controllable temperature profile is obtained, as schematised in the right-hand portion of FIG. 3.

In substance, according to the invention, it is suggested to exploit within the chamber the presence of a metal surface (the deflector plate), of a size proportional to the heating member arranged on the outside of the surface of the sterilisation chamber, which allows to:

concentrate water ebullience for steam generation in a volume ideally separate from the load to be sterilised (the volume located below the plate);

improve steam drying;

increase the kinetic energy of steam, creating turbulence and hence allowing a greater evenness of the heat distribution within the usable chamber of the steam steriliser;

obtain also a partial screening effect, obtaining a temperature decrease of the “hot” wall of the sterilisation chamber in the “critical” areas located in correspondence of the free surface of the water;

reduce temperature irregularities on the lateral wall areas not touched by water, through the generation of a dynamic steam current;

reduce the amount of water injected to carry out a sterilisation cycle, given a certain power and size of a heating member arranged outside in contact with the metal wall of the sterilisation chamber.

Therefore, the chamber arrangement with deflector described here and the relative autoclave obtainable by such chamber perfectly achieve, in an inexpensive and extremely effective way, the objects set forth in the preliminary remarks.

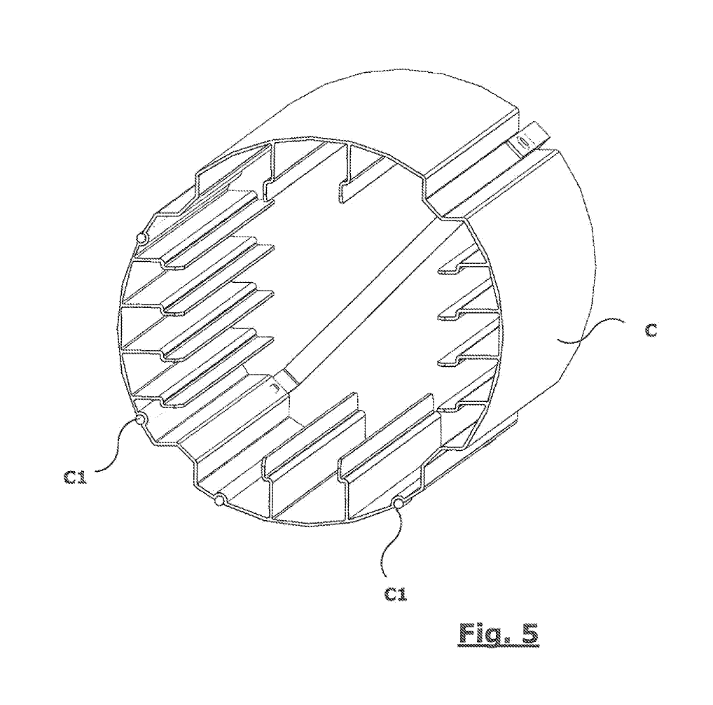

FIG. 5 shows a further embodiment of a deflector integrated in a tray-carrying basket. In particular, a basket C is configured so as to provide a lateral wall the shape of which substantially matches that of the inner surface of the chamber, taking up a tubular, circular shape, as can be appreciated in FIG. 5. Moreover, on the outer surface of the basket spacer means or feet C1 are provided, which guarantee the maintenance of a minimum distance (for example 1-3 mm) between the outer surface of basket C and the inner surface of the chamber. Feet C1 shown in FIG. 5 are shaped as individual members locked on the basket; alternatively, they may be part of a single spacer element to be introduced in the autoclave between the chamber and the very basket to ensure a suitable gap is created.

In this case, too, the presence of the suitably configured basket is exploited, to distribute more evenly the steam and hence the temperature within the chamber. In particular, a gap between the wall chamber and the thin metal wall of the deflector/basket is defined, which forces the steam—which is generated in the lower, overheated areas of the chamber wall—to rise back up, touching the side walls of the basket. By so doing, on the one hand the steam is better dried and distributed, on the other it heats up evenly the basket which in turn distributes evenly the radiated heat.

However, it is understood that the invention is not limited to the particular configuration illustrated above, which represents only a non-limiting example of the scope of the invention, but that a number of variants are possible, all within the reach of a person skilled in the field, without necessarily departing from the scope of the invention as defined in the attached claims.

In particular, it is not ruled out that the deflector may also be discontinuous, i.e. provided with small apertures or flaps which may assist the distribution evenness of steam and hence of temperature in the chamber, for example in correspondence of peculiar shapes of the tray-carrying basket.

Again, it is to be notices that the arrangement of the invention is applicable to any water-injection autoclave (for example with sterilisation cycle having gravity or thermodynamic water ejection), regardless of the fact that in the sterilisation cycle a vacuum pump is employed or not, with or without air-removal and fractioned-vacuum processes.

1) Sterilisation chamber arrangement for an autoclave, comprising a sterilisation container (1) with a conductive wall and a band-shaped heating element, provided with an external heating element (3) arranged at least in the proximity of a lower area of the container wall, means for the injection of water into said container (1) being furthermore provided, characterised in that a deflector plate (2) is furthermore provided arranged inside the container (1) and at short distance from its internal surface at least in the lower portion of the wall.

2) Arrangement as claimed in claim 1), wherein said plate (2) has a complementary curvature with that of the container wall.

3) Arrangement as claimed in claim 1), wherein said plate (2) is arranged at a distance between 1 and 3 mm, preferably about 2 mm from the inner surface of the container.

4) Arrangement as claimed in claim 1), wherein said plate (2) is substantially continuous for the entire depth of the container (1) and extends crosswise for an arc-of-a-circle proportional to the size of the heating element.

5) Arrangement as claimed in claim 1), wherein said plate (2) is fully complementary and joined to a tray-carrying basket.

6) Arrangement as claimed in claim 5), wherein said plate (2) has laterally raised edges (2a) whereto said tray-carrying basket can be fastened.

7) Arrangement as claimed in claim 1), wherein said plate (2) is defined by the thin wall of a tray-carrying basket (C).

8) Tray-carrying basket for an arrangement as claimed in claim 7), comprising a lateral wall substantially shaped like a plate developed on a cylinder and provided with spacer means (C1) apt to define a gap between the same lateral wall of the basket and the inner surface of a sterilisation chamber.

9) Autoclave for the sterilisation of medical tools, characterised in that it comprises an arrangement as claimed in claim 1).

10. Arrangement as claimed in claim 2), wherein said plate (2) is arranged at a distance between 1 and 3 mm, preferably about 2 mm from the inner surface of the container.

11. same Arrangement as claimed in claim 2), wherein said plate (2) is substantially continuous for the entire depth of the container (1) and extends crosswise for an arc-of-a-circle proportional to the size of the heating element.

12. Arrangement as claimed in claim 2), wherein said plate (2) is fully complementary and joined to a tray-carrying basket.

13. Arrangement as claimed in claim 2), wherein said plate (2) is defined by the thin wall of a tray-carrying basket (C).
